(19) United States
(12) Patent Application Publication
(10) Pub. No.: US 2008/0167447 A1
(43) Pub. Date: Jul. 10, 2008

(54) Extraction Of Gelatin

(76) Inventor: Bhanu Manickavasagam, Queensland (AU)

Correspondence Address: Pearl Cohen Zedek Latzer, LLP, 1500 Broadway, 12th Floor, New York, NY, 10036, US

(21) Appl. No.: 11/885,191

(22) PCT Filed: Feb. 28, 2005
(86) PCT No.: PCT/AU2005/000274

§ 371 Date: Sep. 10, 2007

(51) Int. Cl. C07K 1/14; C07K 14/435

(52) U.S. Cl. 530/355; 530/354

(57) ABSTRACT

A process for extracting gelatin from a marine invertebrate, comprising the steps of: 1) pre-treating a collagen-containing portion of the marine invertebrate with an alkali; and 2) extracting gelatin from the pre-treated collagen-containing portion with a weak acid solution at a temperature sufficient for conversion of collagen to gelatin to be effected.

### TECHNICAL FIELD

The present invention is concerned with a process for obtaining gelatin through extraction from marine invertebrates. The gelatin obtained is both a novel protein and an alternative product to land animal gelatin due to the current concerns about Bovine Spongiform Encephalopathy (BSE) or Mad Cow Disease.

### BACKGROUND ART

BSE is an extremely serious disease of cattle, considered to originate from infected meat and bone meal in cattle feed concentrates. BSE is transmissible in cattle, and was first identified in United Kingdom in 1986. It is invariably fatal. There is no treatment and it is difficult to detect. Recent research indicates that humans who eat infected meat could develop Creutzfeldt-Jacob Disease (CJD), the human equivalent of the cattle disease. At least 10 CJD patients in Britain are believed to have contracted the disease from eating beef. Most people who develop CJD are aged between 50 and 70.

Currently the culling of the cattle is of primary importance in the United Kingdom and Europe to safeguard the herd. Nevertheless, BSE poses a significant threat to the future supply of bovine meat and dairy products for the human and animal food chains, and to the supply of important bovine by-products used in the pharmaceutical, medical and cosmetic industries. Presently, the manufacturers of pharmaceuticals across Japan, UK and Europe and other countries have stopped using British beef and beef products in the manufacture of pharmaceuticals and medicines as well as cosmetics products to prevent the spread of “Mad Cow” disease to humans. Also imports of medicine and cosmetics containing substances from British cows have stopped.

The most widely used beef product is collagen. Collagen is a fibrous protein which comprises most of the white fibre in the connective tissues of mammals, particularly the skin, tendon, bone and muscles. A number of different vertebrate collagens have been identified. Indeed, up to 19 groups so far have been identified in vertebrates (Prockop and Kivirikko, 1995) of which type I, II and III represent the most widely distributed species. Collagen comprises about 30% of the total organic matter in mammals and nearly 60% of the protein content. Collagen is deposited rapidly during periods of rapid growth, and its rate of synthesis declines with age, particularly in tissues that undergo little remodeling.

The collagen molecule is built from three peptide chains which are helical in conformation. The helix extends through 1014 residues per chain (Hoffmann et al 1980). At the end of the triple helical domain, short non-helical chains, namely telopeptides, having a non-repeating sequence and spanning from 9 to 25 residues, extend beyond the triple helix from both ends of each chain (Hoffman et al, 1980). The telopeptide portions of native collagen are believed to be the major sites of its immunogenicity and have been shown to play a crucial role in directing fibrillogenesis (Helseth and Veis 1981). The length of the helix and the nature and size of nonhelical portions of the molecule vary from type to type. If the triple helical structure of the collagen molecule is destroyed by heat, the properties of the polypeptides change entirely in spite of having the same chemical composition.

In skin, collagen exists as fibres which are woven into networks constituting fibre bundles, the fibres being maintained in the bundle by interfibrillar cement. Collagen fibrils typically have a length of about 2 mm while the fibres are naturally much longer and of greater diameter.

Vertebrate collagen has a molecular weight of 300,000 Daltons. Each strand of the triple helix has a molecular weight of approximately 100,000 Daltons and assumes a left-handed helix configuration. Most vertebrate collagens present in skin, tendon, muscle, and bone are composed of two identical and one different α chains denoted by [(α1)₂α2] (Piez et al. 1963; Lewis and Piez, 1964; Miller et al, 1967; McClain et al. 1970) except for codfish skin and chick bone collagen which contains three different chains [(α1) (α2) (α3)] (Piez, 1965; Francois and Glincher 1967). Cartilage collagen has in addition to molecules of chain composition [(α1)₂α2], another type of molecule which is composed of three identical chains, [α1(II)₃] (Miller 1971; Trelstad 1970). The α1 (II) chain is apparently different from the α1 chain, which is designated α1(I) only when compared to α1 (II), in its high content of glycosylated hydroxylysines. The collagen present in basement membranes (Kefalides, 1971) and sea anemone body wall (Katzman and Kang 1972) have also been confirmed to consist of identical α chains.

Collagen is the only mammalian protein containing large amounts of hydroxyproline and it is extraordinarily rich in glycine (approximately 30%) and proline. The hydroxyproline is essential for the formation of hydrogen-bonded water-bridges through the hydroxyl group and the peptide chain, thereby stabilising the triple helix. In soluble collagen the inter-molecular bonds have been cleaved, but leaving the triple helices intact.

Gelatin is another very important biopolymer that has found widespread use in the food, pharmaceutical and photographic industries over the years. Traditionally it occurs as a transparent dessert jelly, but is widely used in confectionery, jellied meats and chilled dairy products.

Gelatin is a protein derived from collagen. The source and type of collagen will influence the properties of the resulting gelatin. The amino acid content and sequence varies from one source to another but always consists of large amounts of proline, hydroxyproline and glycine. Since most of the commercial gelatins are obtained from either pigskin or cowhide, there has been considerable interest in pursuing alternative substitutes. This has especially been the case since the recent BSE (bovine spongiform encephalopathy) crisis.

When collagen is heated at a certain temperature the collagen molecule undergoes a helix coil transition. The helix unfolds and the collagen becomes more readily soluble. The temperature at which this occurs depends upon the amount of proline and hydroxyproline in the α chain, and this temperature is the point of denaturing. For deep cold water fish collagen, this temperature is approximately 15° C. while for bovine collagen it is approximately 40° C. At a certain temperature the collagen in the raw skin will relax and the skin will shrink (shrinkage temperature). The amount of imino acids, proline and hydroxyproline, determines the shrinkage temperature and the denaturing temperature.

From vertebrates, the raw materials used for the manufacture of gelatin are pigskin, cattle hides, and cattle bones. The processing of gelatin from these raw materials involves numerous steps and the yields are low. Severe processing is required to solubilise gelatins from stable highly cross-linked ossein (crushed, acid-demineralized and degreased bone) and cattle hide. Gelatins derived from these sources are almost fully deamidated and have isoelectric points close to pH 5.

From vertebrates, the extraction of gelatin depends upon both dissolving and hydrolysing the denatured skin. The gelatin may retain some covalent bonds between alpha chains, which would give rise to multiples of single chain lengths of 95,000 Daltons.

The melting and gelling temperature of gelatin has been found to correlate with the proportion of the imino acids, proline and hydroxyproline in the original collagen (Veis 1964). This is typically approximately 24% for mammals and 16-18% for most fish species.

Thus the conversion of insoluble collagen to soluble gelatin constitutes the essential transformation in gelatin manufacture. The properties of the gelatin depend to a great extent on the raw material employed, on the decomposition process selected, and especially on the reaction conditions during decomposition, extraction, and drying.

International Publication WO02/102831 describes a process for isolating collagen from a marine invertebrate through treating a collagen-containing portion of the marine invertebrate with a weak acid solution in order to solubilise the collagen. The collagen fraction may be heated to convert it to gelatin, but it has been found that the gelatin product obtained through this process varies greatly in quality. Accordingly, there remains no satisfactory way to extract gelatin from a non-bovine source.

### DISCLOSURE OF THE INVENTION

According to a first aspect of the present invention there is provided a process for extracting gelatin from a marine invertebrate, comprising the steps of:
1) pre-treating a collagen-containing portion of the marine invertebrate with an alkali; and
2) extracting gelatin from the pre-treated collagen-containing portion with a weak acid solution at a temperature sufficient for conversion of collagen to gelatin to be effected.

Typically the gelatin is isolated and purified following extraction. The gelatin extraction slurry may be centrifuged, and the pellet stored for further extraction. The gelatin extract supernatant may be purified, for example, by buffer exchange against deionised water. Advantageously, the purified gelatin solution is freeze-dried for storage.

Advantageously, the alkali used for pre-treatment is a dilute alkali solution. The alkali may be any suitable alkali and, for convenience, the very common and cheap bases, sodium hydroxide and potassium hydroxide, are preferred. Advantageously, the dilute solution has a concentration between 0.001M and 0.5M, more preferably, between 0.01M and 0.1M and most preferably 0.02M.

Typically, the collagen-containing portion is pre-treated with a sufficient volume of dilute alkali solution to allow all of it to come into intimate admixture therewith. The collagen-containing portion is typically the meat from an invertebrate such as abalone, and a convenient volume of dilute alkali solution is between 1 and 10 litres per kilogram of meat, preferably 6 litres per kilogram of meat.

Advantageously, the meat has been blended prior to the pre-treatment, therefore the volumes above are per kilogram of blended meat in this embodiment of the invention.

A typical mixing time with blended meat is between 10 minutes and 12 hours, more preferably between 30 minutes and 2 hours, and most preferably 60 minutes.

The temperature at which pre-treatment takes place will be below the temperature at which conversion from collagen to gelatin takes place. Typically the pre-treatment will be performed at less than 50° C., preferably at less than 40° C. and most preferably at room temperature.

Following pre-treatment it is typical to centrifuge the pre-treatment slurry and isolate the pelleted tissue for extraction, which may be preceded with a washing step.

In a particularly preferred embodiment of the invention gelatin is extracted from the washed pelleted tissue with an acetic acid solution, typically a 3% solution at pH 4. A weak acid is one with a dissociation constant between 1.0×10⁻⁵ and 1.0×10⁻² in aqueous solution and so is predominantly un-ionised. Suitable weak acids may be readily identified by the person skilled in the art, but include lactic, butyric, formic, propionic and citric acids.

The volume of weak acid solution used in the extraction step is typically 1 to 10 litres, preferably 6 litres per kilogram of meat. The extraction step will typically proceed for a period between 10 minutes and one week, preferably between 1 hour and 48 hours, more preferably between 2 and 24 hours and preferably for 9 hours. The gelatin extraction step takes place at a temperature where a conversion from collagen to gelatin will be effected. For abalone the temperature will be in excess of 50° C. in order for sufficient conversion of collagen to gelatin to take place and below 100° C. to ensure that excessive denaturation of gelatin does not occur. Typically the extraction of gelatin takes place at between 55 and 65° C., most preferably at 60° C.

Typically the marine invertebrate is prepared for extraction by mechanical disruption of the collagen-containing portion.

Advantageously, the collagen-containing portion is muscle tissue, which has preferably had pigment removed therefrom. This may be achieved by soaking the intact muscle tissue in a weak acid solution. The weak acid solution is typically an acetic acid solution, preferably a 0.2M solution.

In a particularly preferred embodiment invention, the marine invertebrate is abalone.

Preferably, the abalone is a commercial species such as the Black-lip abalone, Haliotis ruber, the Brown-lip abalone, Haliotis conicopora and the Green-lip abalone, Haliotis laevigata, or Roe's abalone, Haliotis roei.

According to a second aspect of the invention there is provided gelatin when prepared by the process described above.

### BRIEF DESCRIPTION OF THE DRAWINGS

FIG. 1 is an SDS-PAGE analysis comparing abalone gelatin prepared in accordance with example 1 to bovine gelatin and a molecular weight standard. The abalone gelatin is in lane 2, the bovine gelatin in lane 3 and the molecular weight standard in lane 1.

### MODES FOR PERFORMING THE INVENTION

### Example 1

### Extraction of Gelatin from the Whole Muscle Tissue of Abalone

Step 1. Frozen abalone meat was stored at −18° C. on arrival at QBT.
Step 2. Abalone meat was removed from the freezer and thawed as required.
Step 3. The pigmentation from the foot area and adductor area was removed by scrubbing with a stiff bristled brush under running water.
Step 4. The skirt area of the abalone was trimmed off with a scalpel and stored for later use.
Step 5. The mouth area was cut away using a scalpel and stored for later use.
Step 6. The whole muscle tissue was cut into quarters and weighed.
Step 7. The tissue was blended by passage through a Comitrol 3600. Hold-up in the cutting head was flushed through with deionised water. The blended tissue was weighed.
Step 8. The blended tissue was pretreated in 6 L of 0.02M NaOH per kg of blended meat with mixing for 60 minutes at room temperature.
Step 9. The pretreatment slurry was centrifuged at 6000×g for 5 minutes.
Step 10. The pelleted tissue was washed by resuspension in 6 L/kg of deionised water with stirring at room temperature for 10 minutes, followed by centrifugation at 6000×g for 5 minutes.
Step 11. The pelleted tissue was rewashed as in Step 10.
Step 12. The gelatin was extracted from the washed pelleted tissue with 6 L of 3% acetic acid at pH 4.0 per kg of tissue (blended weight) with stirring at 60° C. for 9 hours.
Step 13. The gelatin extraction slurry was centrifuged at 6000×g for 10 minutes. The pellet was stored for later use.
Step 14. The gelatin extract supernatant was buffer exchanged against 5 volumes of deionised water using a 10 kD NMCO membrane.
Step 15. The buffer exchanged gelatin solution was transferred to trays and pre-frozen to −20° C. in the chamber of a freeze dryer.
Step 16. The gelatin was freeze dried to a final product temperature of 20° C. This took approximately 48 hr.
Step 17. The freeze dried gelatin was milled using the Comitrol 3600.
Step 18. The milled gelatin was stored for analysis.

### 2^{nd} Extraction

The pelleted tissue from Step 13 of the 1^{st} extraction was re-extracted in 6 L of 3% acetic acid at pH 4.0 per kg of meat (original blended weight) with stirring at 80° C. for 6 hours.

The process then proceeds as from Step 13 of the 1^{st} extraction.

### Example 2

### Analysis of Freeze Dried Gelatin Analyses of Freeze Dried Gelatin

### 1. Appearance

Note was made of the colour, odour, and texture of the material by visual inspection.

### 2. Yield

The yield of freeze dried product was determined on a weight basis with respect to the initial (pre-blending) abalone meat weight used for the process.

### 3. Solubility

The solubility of freeze dried material was tested at a concentration of 1%. To around 50 mg of freeze dried material, deionised water was added to 10 mg/ml and stirred on a magnetic stirrer at room temperature. The clarity of the solution was observed.

### 4. Protein Estimation

Protein estimation was carried out using the Pierce BCA assay. This method is based on the reduction in alkaline conditions of Cu²⁺, to Cu¹⁺ by protein (buiret reaction) and the colourimetric detection of Cu¹⁺ using bicinchoninic acid (BCA).

An appropriate amount of working reagent was prepared by the mixture of 50 parts of reagent A and 1 part of reagent B. For each sample, 2 ml of working reagent was aliquoted into 5 ml polystyrene tubes.

Freeze dried abalone gelatin samples were redissolved in deionised water. These samples were further diluted in deionised water to a concentration of around 1 mg/ml. Then 0.1 ml of each sample was added to a tube and mixed by gentle inversion. A blank was prepared using 0.1 ml deionised water. The tubes were placed in a preheated water bath at 37° C. for 30 minutes, then allowed to cool on the bench for 10 minutes.

A standard curve was prepared by diluting a stock solution of BSA to a range of concentrations from 25-2000 μg/ml and assaying as described above.

The samples were read on a Biorad Smart Spec 3000 spectrophotometer using the inbuilt BCA protein assay function. This allows the storage of standard curves and automatic calculation of sample concentration. Disposable UV grade PMMA cuvettes were used for absorbance measurement at 562 nm.

### 5. Molecular Weight

The molecular weight of abalone gelatin was evaluated by sodium dodecyl sulphate polyacrylamide gel electrophoresis (SDS-PAGE). 12% Gradipore iGel precast Tris glycine gels were used. SDS-PAGE was performed according to the method of Laemmli (1970).

Freeze dried abalone gelatin was dissolved at 1 mg/ml in deionised water. Samples were then diluted ½ with Gradipore Glycine sample buffer.

The samples were then placed into a boiling water bath for 3 minutes then allowed to cool. The gel was assembled in a Biorad Mini-Protean 3 electrophoresis cell. The inner chamber was filled with SDS glycine running buffer (prepared from 10× running buffer prepared by QBT P/L and stored at room temperature) and the samples loaded with an autopipettor and standard yellow tips. The total protein load per well was 2 μg. A molecular weight marker (Biorad broad range prestained marker) was run with each gel. The outer chamber was filled with running buffer to the level of the wells.

The running conditions were 150 V constant voltage over 60 minutes with an approximate start current of 50 mA. The gel was then removed from the casing and rinsed with water for around 30 seconds. The gel was stained with around 50 ml of Gradipore Gradipure stain (based on colloidal G-250 Coomassie blue) overnight with gentle shaking. The gel was destained with frequent changes of water. Bands were generally visible after 5 minutes with about a day required for complete destaining.

Permanent storage of gels was achieved by drying between cellophane sheets. The destained gels were soaked in a drying solution of 20% methanol and 2% glycerol with gentle shaking for 15 minutes. Two cellophane sheets per gel were wetted in the drying solution for around 30 seconds. The trimmed gel was clamped between the cellophane sheets in a drying frame and left to stand in an open container at room temperature for 2 days. The gel was then pressed for a number of days to prevent curling.

A log plot is made of molecular weight versus distance migrated down the gel for the molecular weight standard and a linear trendline determined using MS Excel. The formula generated can then be used to calculate the molecular weight of the sample bands according to their migration distance.

### 6. Heavy Metals as Lead

Although heavy metals are present in the Earths crust they may also be introduced to the marine environment by anthropogenic activities such as mining, industry and agriculture. Marine organisms accumulate trace metals to body concentrations well above those in seawater. Abalone are known to be very sensitive to heavy metals.

Lead content was measured using Merckoquant analytical test strips. This test only detects ionic lead and not organic compounds of lead.

The test vessel was rinsed with the solution to be tested and filled to the 5 ml mark. Three drops of the reagent solution (acetic acid) were added. The pH of the test solution should be between 2 and 5. The reaction zone of the test strip was dipped into the solution to be tested for 1 second. The reaction zone should be fully wetted. Excess liquid is shaken off the strip. The reaction zone of the test strip is compared to the colour scale after 2 minutes.

### 7. Residual Moisture

The residual moisture content of the freeze dried material was determined by measuring the weight loss on drying.

An amount of freeze dried material was accurately weighed, placed in an oven at 105° C., and left overnight. The sample was removed, cooled in a dessicator, and reweighed. The sample was returned to the oven and drying continued until a constant weight was achieved.

The moisture content was taken to be the loss in weight of the sample as a percentage of the initial weight. Commercial bovine gelatin typically has a moisture content of 10-13%.

### 8. Microbiological Assay

The freeze dried material was tested for the presence of microbial contamination. Assays were performed for E. coli count, and Salmonella count by USP methods.

E. coli Count

Lactose Broth is added to obtain a 1:10 dilution (i.e. add 90 mL of Lactose Broth to a 10 mL sample). Incubate for a period of 24 hours controlled at a temperature between 30° C. and 35° C. If no growth has occurred re-incubate for another 24 hours and if growth is present, mix by gently shaking. If after 48 hrs no growth has occurred the specimen meets the requirements of the test for absence of Escherichia coli.

For bottles showing growth (turbidity), streak a portion of the Lactose Broth on the surface of a pre-poured MacConkey Agar plate by means of an inoculating loop. Cover and invert petri dishes. Incubate for 24 hours controlled at a temperature of 37° C. Upon examination, if none of the colonies appear as brick red Gram negative rods, the specimen meets the requirements of the test for absence of Escherichia coli.

### Salmonella Count

Add required Fluid Lactose Medium to obtain a 1:10 dilution (i.e. add 90 mL of Fluid Lactose Medium to a 10 mL sample). Incubate for a period of 24 hours controlled at a temperature between 30° C. and 35° C. If no growth has occurred re-incubate for another 24 hours and if growth is present, mix by gently shaking. If after 48 hrs no growth has occurred the specimen meets the requirements of the test for absence of Salmonella.

For bottles showing growth (turbidity), pipette 11.0 mL portions into vessels containing, respectively, 10 mL of Fluid Selenite-Cystine Medium and Fluid Tetrathionate Medium. Mix and incubate for 12 to 24 hours. By means of an inoculating loop, streak a portion of each of the Selenite Cystine Broth (CSB) and Tetrathionate Broth (TB) onto pre-poured agar plates. These plates need to be divided in half and labelled appropriately with the sample number and CSB on one half and TB on the other half. The pre-poured agar plates to be used are Brilliant Green Agar (BGA), Xylose-Lysine-Desoxycholate Agar (XLD) and Bismith Sulfite Agar (BSA). Cover and invert the petri dishes.

Incubate BGA and XLD plates for 24 hours controlled at a temperature of 37° C., and BSA for 48 hours controlled at a temperature of 37° C. Upon examination, if none of the colonies conforms to the description in Table 1, the specimen meets the requirements of the test for absence of the genus Salmonella.

TABLE 1
Morphological Characteristic of Salmonella
Species on Selective Agar Media

| | Characteristic Colonial |
|---|---|
| Selective Medium | Morphology |
| Brilliant Green Agar | Small, transparent, |
| | colourless or pink to white |
| | opaque (frequently |
| | surrounded by pink to red |
| | zone) |
| Xylose-Lysine-Desoxycholate | Red, with or without black |
| Agar | centres |
| Bismuth Sulfite Agar | Black or green |

### 9. Gel Strength

The gel strength, or Bloom, is a measure of the weight in grams required to push a 12.5 mm diameter plunger a distance of 4 mm into a 6.67% gelatin solution which has been gelled by cooling at 10° C. for around 18 hours.

### Results

### 1. Apperance of Freeze Dried Abalone Gelatin-(Table 2)

TABLE 2

| Sample | Appearance |
|---|---|
| 1^{st} Extract (60° C.) | off white powder, |
| | negligible odour |
| 2^{nd} Extract (80° C.) | off white powder, |
| | negligible odour |

### 2. Yield of Abalone Gelatin-(Table 3)

TABLE 3

| Sample | Yield (%) |
|---|---|
| 1^{st} Extract (60° C.) | 1.9 |
| 2^{nd} Extract (80° C.) | 3.1 |
| Total | 5.0 |

### 3. Solubility of Freeze Dried Abalone Gelatin-(Table 4)

TABLE 4

| | Solubility at 1% at Room |
|---|---|
| Sample | Temp. |
| 2^{nd} Extract (80° C.) | complete solubilisation |

### 4. Protein Content of Abalone Gelatin-(Table 5)

TABLE 5

| | Protein Concentration by |
|---|---|
| | BCA |
| Sample | (mg/ml) |
| 1^{st} Extract (60° C.) at 5% (50 mg/ml) | 43.5 |

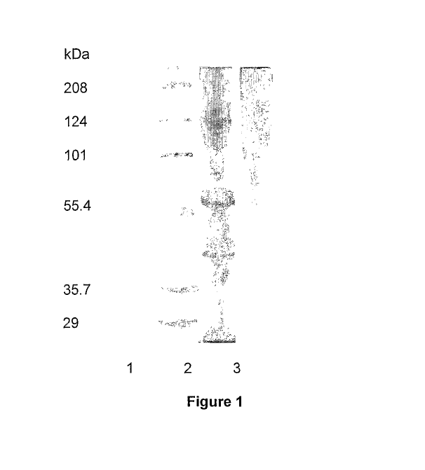

### 5. Molecular Weight of Abalone Gelatin-(FIG. 1, Table 6)

TABLE 6

| Sample | Molecular Weight (kDa) |
|---|---|
| 1^{st} Extract | 20-150 |
| (60° C.) | |

### 6. Heavy Metals Content of Freeze Dried Abalone Gelatin-(Table 7)

TABLE 7

| Sample | Heavy Metals (as Lead) |
|---|---|
| 1^{st} Extract | <20 ppm |
| (60° C.) | |

### 7. Residual Moisture Content of Freeze Dried Abalone Gelatin-(Table 8)

TABLE 8

| Sample | Residual Moisture |
|---|---|
| 1^{st} Extract | 12.7% |
| (60° C.) | |
| 2^{nd} Extract | 13.1% |
| (80° C.) | |

### 8. Microbiological Assay of Abalone Gelatin-(Table 9)

TABLE 9

| Sample | E. coli/ml | Salmonella/ml |
|---|---|---|
| 2^{nd} Extract (80° C.) | none detected | none detected |

### 9. Gel Strength of Abalone Gelatin-(Table 10)

TABLE 10

| Sample | Gel Strength (g) |
|---|---|
| 2^{nd} Extract | 22-65 |
| (80° C.) | |

### REFERENCES

The references listed below have their disclosure incorporated herein through reference:
Francois C. J. and Glincher M. J. (1967) Biochim. Biophys. Acta 133, 91.
Helseth D. L Jr and Veis A (1981) J. Biol. Chem. 256, 7118-7128.
Hofmann H, Fietzek, P. P and Kuhn K (1980) J. Mol Biol. 141, 293-314.
Katzman R. L and Kang A. H (1972) J. Biol. Chem 247, 5486.
Kefalides N. A (1971) Biochem Biophys Res. Commun. 46, 226.
U. K. Laemmili (1970) Nature 227, 680-685.
Laurain G, Delvincourt T, and Szymanowicz A. G. (1980) FEBS Letter, 120, 44-48.
Lewis M. S and Piez K. A. J. (1964) Biol. Chem. 239, 336.
McClain P. E., Creed G. J., Wiley E. R. and Gerrits R. J. (1970) Biochim. Biophys Acta 221, 349.
Miller E. J Biochemistry (1971) 10, 1652.
Miller E. J., Martin G. R., Piez K. A and Powers M. J. J. Biol. Chem (1967) 242, 5481.
Piez K. A Biochemistry (1965) 4, 2590.
Piez, K. A, Eiger A, and Lewis M. S (1963) Biochemistry 2, 58.
Piez K. A (1984) Molecular and aggregate structures of the collagens. In Extracellular Matrix Biochemistry (Piez, K. A and Reddi A. H. eds) pp 1-39, Elsevier N.Y.
Prockop D. J and Kivirikko K. I (1995) Annu. Rev. Biochem 64, 403-434.
Trelstad R. I. Kang A. H Igarashi S. and Gross J. (1970) Biochemistry 9, 4993.
Veis A. (1964) The Gelatin→Collagen Transition. Ch. 5 in The Macromolecular Chemistry of Gelatin, Academic Press, New York and London.

1. A process for extracting gelatin from a marine invertebrate, comprising the steps of:
1) pre-treating a collagen-containing portion of the marine invertebrate with an alkali; and
2) extracting gelatin from the pre-treated collagen-containing portion with a weak acid solution at a temperature sufficient for conversion of collagen to gelatin to be effected.

2. A process as claimed in claim 1 wherein the alkali used for pre-treatment is a dilute alkali solution.

3. A process as claimed in claim 2 wherein the dilute solution has a concentration between 0.001M and 0.5M, more preferably, between 0.01M and 0.1M and most preferably 0.02M.

4. A process as claimed in claim 1 wherein the alkali is sodium hydroxide or potassium hydroxide.

5. A process as claimed in claim 1 wherein the pre-treated collagen-containing portion is centrifuged and washed prior to gelatin extraction.

6. A process as claimed in claim 1 wherein the weak acid solution is an acetic acid solution.

7. A process as claimed in claim 6 wherein the weak acid solution is a 3% acetic acid solution at pH 4.

8. A process as claimed in claim 1 wherein gelatin extraction takes place at a temperature in excess of 50° C. and below 100° C.

9. A process as claimed in claim 8 wherein gelatin extraction takes place at between 55° C. and 65° C.

10. A process as claimed in claim 1 wherein the gelatin extract is centrifuged and buffer exchanged against deionised water.

11. A process as claimed in claim 1 wherein the marine invertebrate is abalone.

12. A process as claimed in claim 11 wherein the abalone is selected from the group consisting of the Black-lip abalone, Haliotis ruber, the Brown-lip abalone, Haliotis conicopora, the Green-lip abalone, Haliotis laevigata and Roe's abalone, Haliotis roei.

13. Gelatin when prepared by the process of claim 1.

14. (canceled)
